(19) United States
(12) Patent Application Publication
(10) Pub. No.: US 2024/0131270 A1
(43) Pub. Date: Apr. 25, 2024

(54) COMPACT RATCHET MECHANISM

(71) Applicant: INSULET CORPORATION, Acton, MA (US)

(72) Inventors: Kyle BREINGAN, Lowell, MA (US); Daniel BARRETT, Somerville, MA (US); Soroush KAMRAVA, Everett, MA (US)

(21) Appl. No.: 18/490,413

(22) Filed: Oct. 18, 2023

Related U.S. Application Data
(60) Provisional application No. 63/380,845, filed on Oct. 25, 2022.

(51) Int. Cl. A61M 5/315

(52) U.S. Cl. CPC A61M 5/31576

(57) ABSTRACT

A drug delivery drive mechanism including a ratchet wheel including a plurality of teeth, a ratchet arm including a first prong and a second prong, wherein the first prong is operable interact with a tooth of the plurality of teeth to rotate the ratchet wheel in a circular direction, and the second prong is operable to interact with another tooth of the plurality of teeth to further rotate the ratchet wheel in the circular direction.

### CROSS-REFERENCE TO RELATED APPLICATION

This application claims priority to and the benefit of U.S. Provisional Application No. 63/380,845, filed Oct. 25, 2022, of which is incorporated herein in its entirety.

### TECHNICAL FIELD

The disclosed embodiments generally relate to medication delivery. More particularly, the disclosed embodiments relate to techniques, processes, systems, and devices that use a compact ratchet mechanism to deliver a medicament to a user.

### BACKGROUND

Wearable drug delivery devices can include a reservoir for storing a liquid drug. A drive mechanism is operated to expel the stored liquid drug from the reservoir for delivery to a user. In some cases, the drive mechanism includes multiple ratchet wheels that provide the necessary angular motion/torque to run the pump. However, based on the arrangement of such ratchet wheels, the drive mechanism may occupy a relatively large volume inside the drug delivery device, increasing the size and cost of the device. In addition, present drive mechanisms utilize a significant portion of the electrical energy stored in the energy storage devices of a wearable drug delivery device.

Accordingly, there is a need for a more power efficient and compact drug delivery device drive mechanism for expelling a liquid drug from a reservoir.

### SUMMARY

At least one aspect of the present disclosure is directed to a drug delivery drive mechanism. The drug delivery drive mechanism includes a ratchet wheel including a plurality of teeth, a ratchet arm including a first prong and a second prong, wherein the first prong is operable interact with a tooth of the plurality of teeth to rotate the ratchet wheel in a circular direction, and the second prong is operable to interact with another tooth of the plurality of teeth to further rotate the ratchet wheel in the circular direction.

Another aspect of the present disclosure is directed to a drug delivery drive mechanism. The drug delivery drive mechanism includes a ratchet wheel including a plurality of teeth and having a center point, a ratchet arm extending from the center point of the ratchet wheel and beyond a perimeter of the ratchet wheel, and a biasing member operable to move the ratchet arm. The ratchet arm includes a prong operable to interact with a tooth of the plurality of teeth of the ratchet wheel and rotate the ratchet wheel in a circular direction, and a biasing member attachment point that is farther from the center point of the ratchet wheel than the prong, and operable to attach the biasing member to the ratchet arm.

Another aspect of the present disclosure is directed to a drug delivery drive mechanism. The drug delivery drive mechanism includes a first ratchet wheel including a first plurality of ratchet wheel teeth and centered about a wheel axis, a second ratchet wheel including a second plurality of ratchet wheel teeth and centered about the wheel axis, a ratchet arm operable to rotate about an arm axis positioned outside a radius of the first ratchet wheel and second ratchet wheel. The ratchet arm includes a first prong operable to interact with a tooth of the first plurality of first ratchet wheel teeth of the first ratchet wheel and rotate the first ratchet wheel in a circular direction, and a second prong operable to interact with a tooth of the second plurality of second ratchet wheel teeth of the second ratchet wheel and rotate the second ratchet wheel in the circular direction.

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1 illustrates a schematic diagram of a drug delivery system according to embodiments of the present disclosure;

FIG. 2A illustrates a schematic diagram of a drug delivery system according to embodiments of the present disclosure;

FIG. 2B illustrates a perspective view of an example of a drug delivery system of FIG. 2A according to embodiments of the present disclosure;

FIG. 2C illustrates a perspective view of an example of a drive mechanism of a drug delivery system of FIG. 2A according to embodiments of the present disclosure;

FIG. 2D illustrates a perspective view of the example drive mechanism of FIG. 2C according to embodiments of the present disclosure;

FIG. 3 illustrates a drive mechanism of a liquid drug delivery device according to embodiments of the present disclosure;

FIG. 4 illustrates a ratchet mechanism of a drive mechanism according to embodiments of the present disclosure;

FIGS. 5A, 5B illustrate an example operation of a ratchet mechanism of FIG. 4 according to embodiments of the present disclosure;

FIG. 6 illustrates a ratchet mechanism of a drive mechanism according to embodiments of the present disclosure;

FIGS. 7A, 7B illustrate an example operation of a ratchet mechanism of FIG. 6 according to embodiments of the present disclosure;

FIG. 8 illustrates a ratchet mechanism of a drive mechanism according to embodiments of the present disclosure;

FIG. 9 illustrates a ratchet mechanism of a drive mechanism according to embodiments of the present disclosure;

FIGS. 10A, 10B illustrate an example operation of a ratchet mechanism of FIG. 9 according to embodiments of the present disclosure;

FIG. 11 illustrates a variation of a ratchet mechanism of FIG. 9 according to embodiments of the present disclosure;

FIG. 12 illustrates a ratchet mechanism of a drive mechanism according to embodiments of the present disclosure; and

FIG. 13 illustrates a ratchet mechanism of a drive mechanism according to embodiments of the present disclosure.

The drawings are not necessarily to scale. The drawings are merely representations, not intended to portray specific parameters of the disclosure. The drawings are intended to depict exemplary embodiments of the disclosure, and therefore are not to be considered as limiting in scope. Furthermore, certain elements in some of the figures may be omitted, or illustrated not-to-scale, for illustrative clarity. Still furthermore, for clarity, some reference numbers may be omitted in certain drawings.

### DETAILED DESCRIPTION

Systems, devices, and methods in accordance with the present disclosure will now be described more fully hereinafter with reference to the accompanying drawings, where one or more embodiments are shown. The systems, devices, and methods may be embodied in many different forms and are not to be construed as being limited to the embodiments set forth herein. Instead, these embodiments are provided so the disclosure is thorough and complete, and fully conveys the scope of methods and devices to those skilled in the art. Each of the systems, devices, and methods disclosed herein provides one or more advantages over conventional systems, components, and methods.

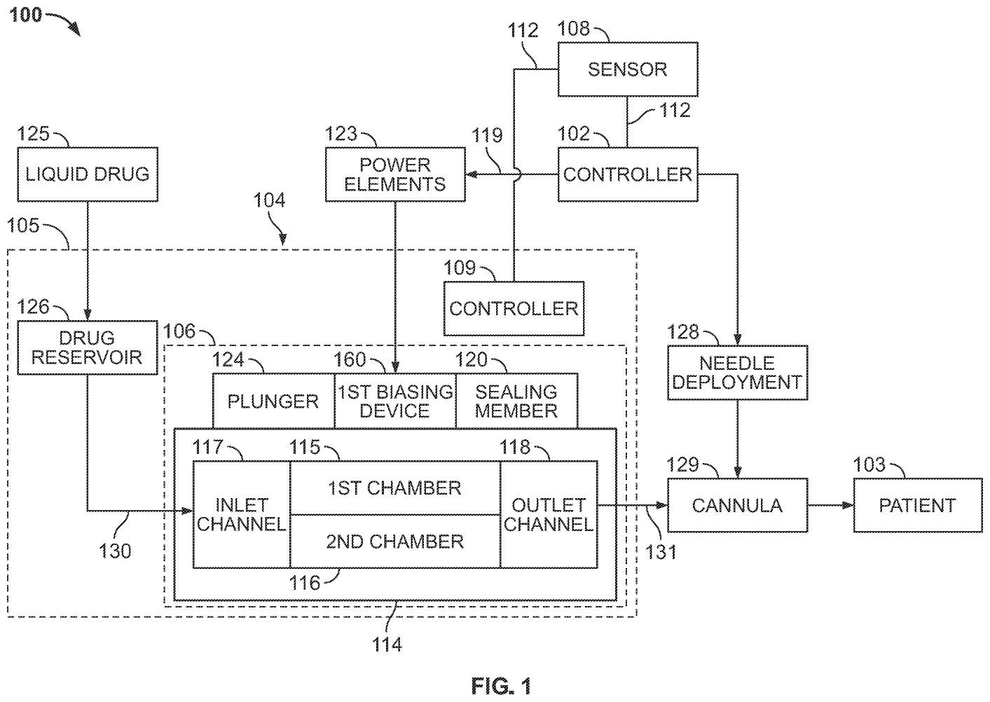

FIG. 1 illustrates a simplified block diagram of an example system 100. The system 100 may be a wearable or on-body drug delivery device and/or an analyte sensor attached to the skin of a patient 103. The system 100 may include a controller 102, a pump mechanism 104 (hereinafter “pump 104”), and a sensor 108. The sensor 108 may be one or more of a glucose or other analyte monitor such as, for example, a continuous glucose monitor, a ketone sensor, a heart rate monitor, a blood oxygen sensor element, or the like, and may be incorporated into the wearable device. The sensor(s) 108 may, for example, be operable to measure blood glucose (BG) values of a user to generate a measured BG level signal 112. The controller 102, the pump 104, and the sensor(s) 108 may be communicatively coupled to one another via a wired or wireless communication path. For example, each of the controller 102, the pump 104 and the sensor(s) 108 may be equipped with a wireless radio frequency transceiver operable to communicate via one or more communication protocols, such as Bluetooth®, or the like. As described in greater detail herein, the system 100 may also include a pump 104 which includes a drive mechanism 106 having at least one housing 114 defining a pump chamber 115, a channel chamber 116, an inlet channel 117, and an outlet channel 118. The drive mechanism 106 may further include a resilient sealing member 120 enclosing the pump chamber 115, and a first biasing device 160 (e.g., smart memory alloy (SMA) wire, shape memory alloy wire, or the like) operable to bias or move a plunger 124 relative to the pump chamber 115, as described in greater detail herein. In an example, the drive mechanism 106, the controller 109 and the drug reservoir 126 may be incorporated in a housing 105, which may be one or more parts that may be a combination of reusable and disposable parts. The housing 105 may be formed from one or more materials, such as a plastic, a metal or the like. The system 100 may include additional components not shown or described for the sake of brevity.

The controller 102 may receive a desired BG level signal, which may be a first signal, indicating a desired BG level or range for the patient 103. The desired BG level or range may be stored in memory of a controller 109 on pump 104, received from a user interface of the controller 102, or another device, or by an algorithm within controller 109 (or controller 102). The sensor(s) 108 may be coupled to the patient 103 and operable to measure an approximate value of a BG level of the user. In response to the measured BG level or value, the sensor(s) 108 may generate a signal indicating the measured BG value. As shown in the example, the controller 102 may also receive from the sensor(s) 108 via a communication path, the measured BG level signal 112, which may be a second signal.

Based on the desired BG level and the measured BG level signal 112, the controller 102 or controller 109 may generate one or more control signals for directing operation of the pump 104. For example, one control signal 119 from the controller 102 or controller 109 may cause the pump 104 to turn on, or activate one or more power elements 123 operably connected with the pump 104. In the case where the first biasing device 160 is an SMA wire, activation of the SMA wire by the power element 123 may cause the SMA wire to change shape and/or length, which in turn may cause movement of the plunger 124 and the resilient sealing member 120. The specified amount of a liquid drug 125 (e.g., insulin, GLP-1, pramlintide, or a co-formulation of insulin, GLP-1 or pramlintide; a chemotherapy drug; a blood thinner; a pain medication; an arthritis drug; or the like) may be drawn from the reservoir 126 into the pump chamber 115, through the inlet channel 117, in response to a change in pressure due to the change in configuration of the resilient sealing member 120 and the plunger 124. In some examples, the specified amount of the liquid drug 125 to be delivered may be determined based on a difference between the desired BG level and the actual BG level signal 112. For example, specified amount of the liquid drug 125 may be determined as an appropriate amount of insulin to drive the measured BG level of the user toward the desired BG level. Based on operation of the pump 104, as determined by the control signal 119, the patient 103 may receive the liquid drug from a reservoir 126. The system 100 may operate as a closed-loop system, an open-loop system, or as a hybrid system. In an exemplary closed-loop system, the controller 109 may direct operation of the pump 104 without input from the user or controller 102, and may receive BG level signal 112 from the sensor(s) 108. The sensor(s) 108 may be housed within the pump 104 or may be housed in a separate device and communicate wirelessly directly with the pump 104 (e.g., with controller 109) or with an external controller 102.

As further shown, the system 100 may include a needle deployment component 128 in communication with the controller 102 or the controller 109. Though shown separately, needle deployment component 128 may be integrated within pump 104. The needle deployment component 128 may include a needle and/or cannula 129 deployable into the patient 103 and may have one or more lumens and one or more holes at a distal end thereof. The cannula 129 may form a portion of a fluid path coupling the patient 103 to the reservoir 126. More specifically, the inlet channel 117 may be coupled to the reservoir 126 by a first fluid path component 130. The first fluid path component 130 may be of any size and shape and may be made from any material. The first fluid path component 130 enables fluid, such as the liquid drug 125 in the reservoir 126, to be transferred to the drive mechanism 106.

As further shown, the outlet channel 118 may be coupled to the cannula 129 by a second fluid path component 131. The second fluid path component 131 may be of any size and shape and may be made from any material. The second fluid path component 131 may be connected to the cannula 129 to allow fluid expelled from the pump 104 to be provided to the patient 103. The first and second fluid path components 130 and 131 may be rigid, flexible, or a combination thereof.

The controller 102/109 may be implemented in hardware, software, or any combination thereof. The controller 102/109 may, for example, be a processor, a logic circuit or a microcontroller coupled to a memory. The controller 102/109 may maintain a date and time as well as provide other functions (e.g., calculations or the like) performed by processors. The controller 102/109 may be operable to execute an artificial pancreas (AP) algorithm stored in memory (not shown in this example) that enables the controller 102/109 to direct operation of the pump 104. For example, the controller 102/109 may be operable to receive an input from the sensor(s) 108, wherein the input comprises analyte level data, such as blood glucose data or levels over time. Based on the analyte level data, the controller 102/109 may modify the behavior of the pump 104 and resulting amount of the liquid drug 125 to be delivered to the patient 103.

The power elements 123 may be a battery, a supercapacitor, a piezoelectric device, or the like, for supplying electrical power to the pump 104. In other embodiments, the power element 123, or an additional power source (not shown), may also supply power to other components of the pump 104, such as the controller 102, memory, the sensor(s) 108, and/or the needle deployment component 128.

In an example, the sensor(s) 108 may be a device communicatively coupled to the controller 102 and may be operable to measure a blood glucose value at a predetermined time interval, such as approximately every 5 minutes, 1 minute, or the like. The sensor(s) 108 may provide a number of blood glucose measurement values to the AP application.

In some embodiments, the pump 104, when operating in a normal mode of operation, provides insulin stored in the reservoir 126 to the patient 103 based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like) provided by the sensor(s) 108 or other functional elements of the system 100 or pump 104. For example, the pump 104 may contain analog and/or digital circuitry that may be implemented at the controller 102/109 for controlling the delivery of the drug or therapeutic agent. The circuitry used to implement the controller 102/109 may include discrete, specialized logic and/or components, an application-specific integrated circuit, a microcontroller or processor that executes software instructions, firmware, programming instructions or programming code enabling, for example, an AP application stored in memory, or any combination thereof. For example, the controller 102/109 may execute a control algorithm and other programming code that may make the controller 102/109 operable to cause the pump to deliver doses of the drug or therapeutic agent to a user at predetermined intervals or as needed to bring blood glucose measurement values to a target blood glucose value. The size and/or timing of some of the doses may be pre-programmed, for example, into the AP application by the patient 103 or by a third party (such as a health care provider, a parent or guardian, a manufacturer of the wearable drug delivery device, or the like) using a wired or wireless link, or may be calculated iteratively by the controller 102 or controller 109, such as every 5 minutes.

Although not shown, in some embodiments, the sensor(s) 108 may include a processor, memory, a sensing or measuring device, and a communication device. The memory of the sensor(s) 108 may store an instance of an AP application as well as other programming code and be operable to store data related to the AP application.

In various embodiments, the processor of the sensor(s) 108 may include discrete, specialized logic and/or components, an application-specific integrated circuit, a microcontroller or processor that executes software instructions, firmware, programming instructions stored in memory, or any combination thereof.

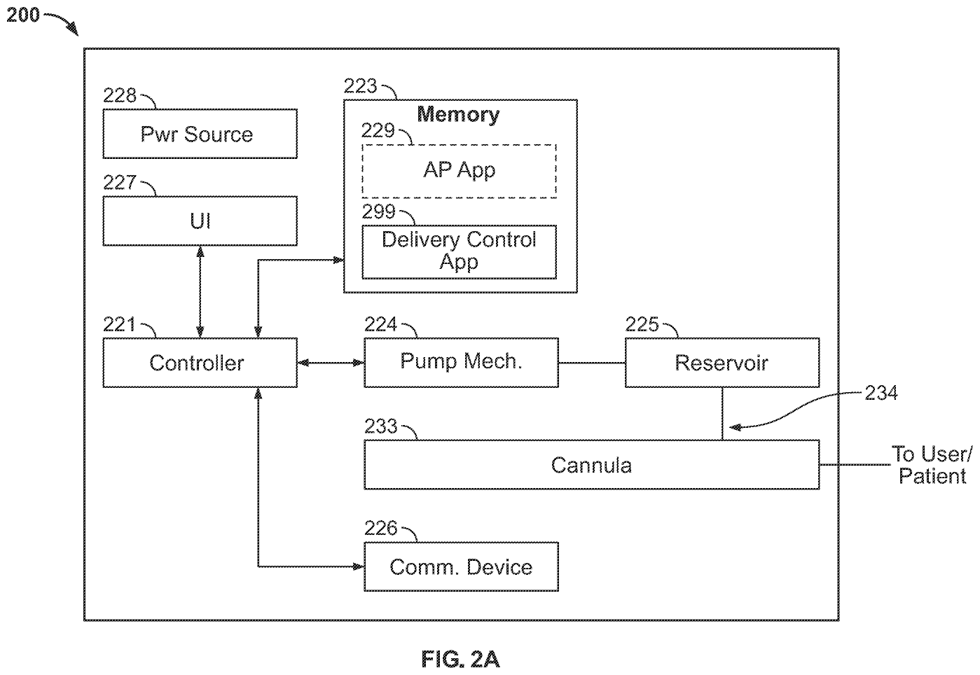

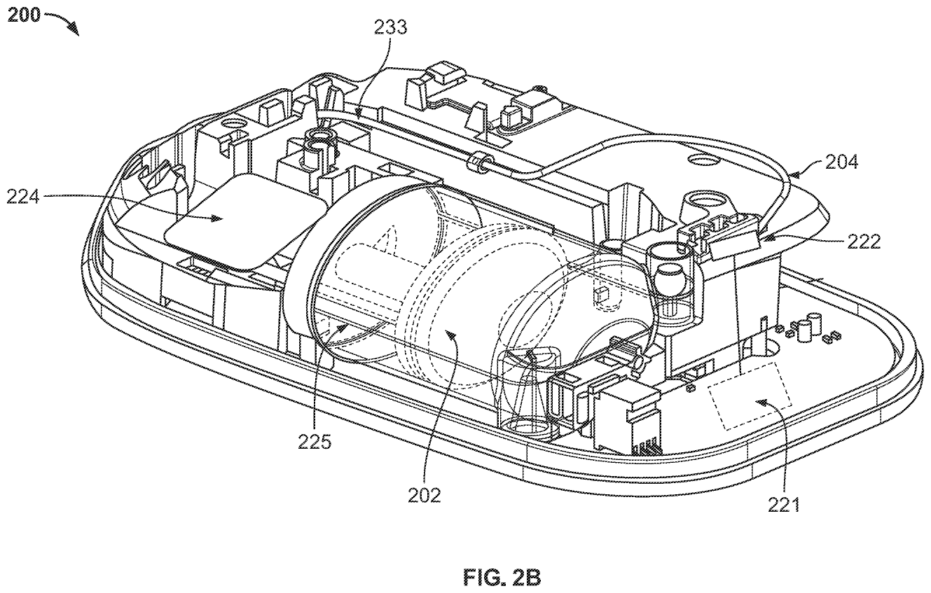

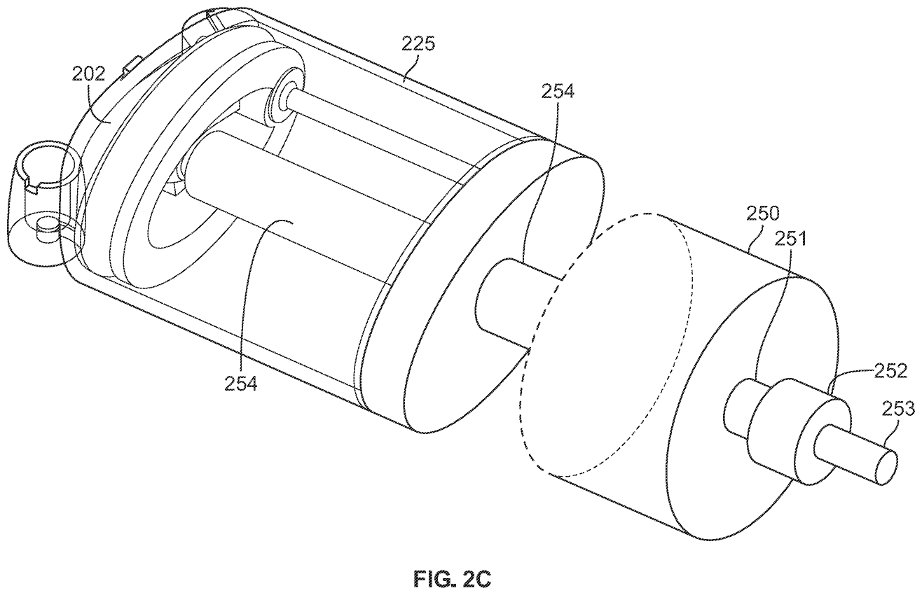

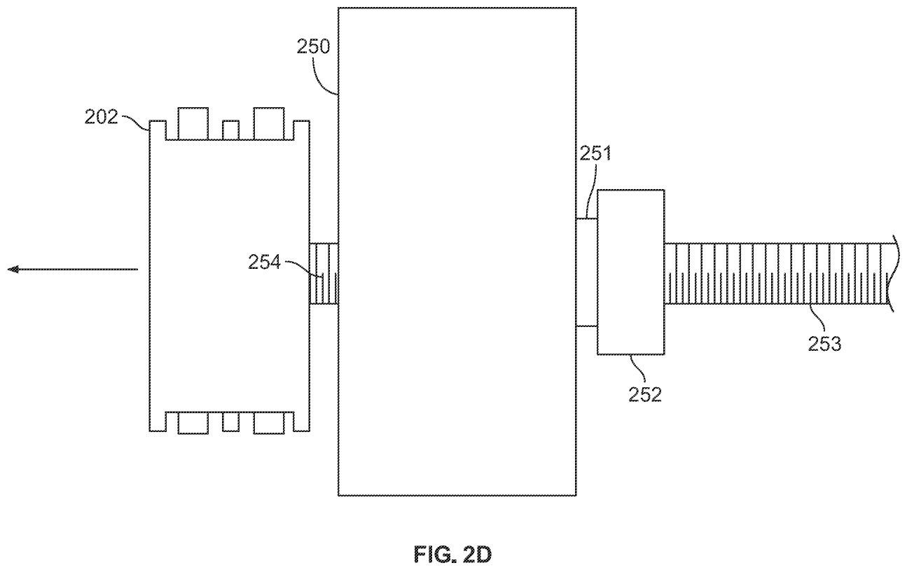

FIG. 2A illustrates a simplified block diagram of another example system 200. The system 200 may include a controller 221, a memory 223, an AP application 229 and delivery control application 299 stored in the memory 223, a pump mechanism 224, a communication device 226, user interface 227, and a power source 228. The memory 223 may be operable to store programming code and applications including a delivery control application 299, the AP application 229 and data. The delivery control application 299 and the AP application 229 may optionally be stored on other devices.

The controller 221 may be coupled to the pump mechanism 224 and the memory 223. The controller 221 may include logic circuits, a clock, a counter or timer as well as other processing circuitry, and be operable to execute programming code and the applications stored in the memory 223 including the delivery control application 299. A communication device 226 may be communicatively coupled to the controller 221 and may be operable to wirelessly communicate with an external device, such as a personal diabetes management device, a smart device such as a smartphone and/or a smartwatch, or the like.

The pump mechanism 224 may be operable to deliver a drug, like insulin, at a fixed or variable rate. For example, an AP application or AID algorithm executing on a personal diabetes management device or a smart phone may determine or be informed that a user's total daily insulin (e.g., bolus and basal deliveries) is 48 units per 24 hours, which may translate to an exemplary physiological basal dosage rate of 1 unit per hour (48/24/2 (assuming a 1:1 basal/bolus ratio)) that may be determined according to a diabetes treatment plan. Of course, the pump mechanism 224 may be operable to deliver insulin at rates different from the example physiological dosage rate of 1 unit per hour. In an example, the system 200 may be attached to the body of a user, such as a patient or diabetic via, for example, by an adhesive, (e.g., directly attached to the skin of the user) and may deliver any therapeutic agent, including any drug or medicine, such as insulin, morphine, or the like, to the user. In an example, a surface of the system 200 may include an adhesive (not shown) to facilitate attachment to a user. The system 200 may, for example, be worn on a belt or in a pocket of the user and the liquid drug may be delivered to the user via tubing to an infusion site on the user.

In various examples, the system 200 may be an automatic, wearable drug delivery device. For example, the system 200 may include a reservoir 225 configured to hold a liquid drug (such as insulin), a needle and/or cannula 233 for delivering the drug into the body of the user (which may be done subcutaneously, intraperitoneally, or intravenously), and a pump mechanism 224, or other drive mechanism, for transferring the drug from the reservoir 225, through a needle or cannula 233, and into the user.

The pump mechanism 224 may be fluidly coupled to reservoir 225, and communicatively coupled to the medical device controller 221. The pump mechanism 224 may be coupled to the reservoir 225 and operable to output the liquid drug from the reservoir 225 via a fluid delivery path and out of the cannula 233. The pump mechanism 224 may have mechanical parameters and specifications, such as a pump resolution, that indicate mechanical capabilities of the pump mechanism. The pump mechanism 224 may also have electrical connections to control circuitry (not shown) that is operable to control operation of the pump mechanism 224. The pump resolution is a fixed amount of insulin the pump mechanism 224 delivers in a pump mechanism pulse, which is an actuation of the pump mechanism for a preset time period. Actuation may be when power from the power source 228 is applied to the control circuitry coupled to the pump mechanism 224 and the pump mechanism 224 operates to pump a fixed amount of insulin in a preset amount of time from the reservoir 225. Alternatively, the pump mechanism 224 may be substantially mechanical in structure and operation and utilize mechanical energy storage devices, such as springs or other biasing members to operate the pump mechanism 224.

The cannula 233 of FIG. 2A may be coupled to the reservoir 225 via a fluid delivery path 234. The cannula 233 may be operable to output the liquid drug to a user when the cannula 233 is inserted in the user.

The system 200 may also include a power source 228, such as a battery, a supercapacitor, a piezoelectric device, or the like, that is operable to supply electrical power to the pump mechanism 224 and/or other components (such as the controller 221, memory 223, and the communication device 226) of the system 200.

As shown in FIG. 2B, the system 200 may include a plunger 202 positioned within the reservoir 225. An end portion or stem of the plunger 202 can extend outside of the reservoir 225. The pump mechanism 224 may, under control of the controller 221, be operable to cause the plunger 202 to expel the fluid, such as a liquid drug (not shown) from the reservoir 225 and into a fluid component 204 and cannula 233 by advancing into the reservoir 225. In various examples, a pressure sensor, such as that shown at 222, may be integrated anywhere along the overall fluid delivery path of the system 200, which includes the reservoir 225, the fluid delivery path component 204, and the cannula 233.

The controller 221 may be implemented in hardware, software, or any combination thereof. In various examples, the controller 221 can be implemented as dedicated hardware (e.g., as an application specific integrated circuit (ASIC)). The controller 221 may be a constituent part of the system 200, can be implemented in software as a computational model, or can be implemented external to the system 200 (e.g., remotely). The controller 221 may be configured to communicate with one or more sensors (e.g., sensor(s) 108 of FIG. 1).

As described above, a reservoir, such as 225, may be included in a drug delivery device to store a liquid drug (e.g., insulin). For example, the reservoir 225 may be filled, or partially filled, with a liquid drug or a liquid drug solution. In one example, a liquid drug solution is a mixture of the liquid drug and added preservatives. The reservoir may store the liquid drug until all of the liquid drug has been dispensed (e.g., into a patient via a cannula). As such, the liquid drug (or solution) may remain in the reservoir for a period of time (e.g., 1 day, 3 days, 1 week, 2 weeks, etc.).

FIG. 2C illustrates an example of a reservoir coupled to a drive mechanism 250 that may be included in the pump mechanism 224. Likewise, FIG. 2D illustrates a perspective view of the drive mechanism 250. As disclosed in later examples, the drive mechanism 250 (shown in more detail in later examples) may include a drive wheel, co-planar ratchet wheels, and an actuator. In one example, the set of ratchet wheels are attached to the drive wheel. The drive wheel may be coupled to a plunger 202 via an elongated shaft 254. At a high level, the ratchet wheels are engaged by the actuator to incrementally drive the drive wheel and advance the plunger 202 and the elongated shaft 254 into the reservoir 225. The elongated shaft 245 advances the plunger 202 to dispense the liquid drug out of the reservoir 225. In one example, a pump mechanism coupling 251 is operable to connect either the first ratchet wheel or the second ratchet wheel of the drive mechanism 250 to a drive element 252. The drive element 252 includes (or is otherwise coupled to) a lead screw 253 that is coupled to the plunger 202 (e.g., via the elongated shaft 254). The pump mechanism 224 is operable to rotate the lead screw 253 (e.g., via the drive mechanism 250) and move the plunger 202 to expel the liquid drug from the reservoir 225. In some examples, the rotation of the lead screw 253 causes the elongated shaft 254 to advance within the reservoir 225.

Improved drive mechanisms for liquid drug delivery devices are provided herein. In at least one embodiment, the drive mechanism includes a ratchet wheel including a plurality of teeth and a ratchet arm including a first prong and a second prong. In some examples, the first prong is operable to interact with a tooth of the plurality of teeth to rotate the ratchet wheel in a circular direction and the second prong is operable to interact with another tooth of the plurality of teeth to further rotate the ratchet wheel in the circular direction. Such an arrangement enables the drive mechanism to have a compact form factor.

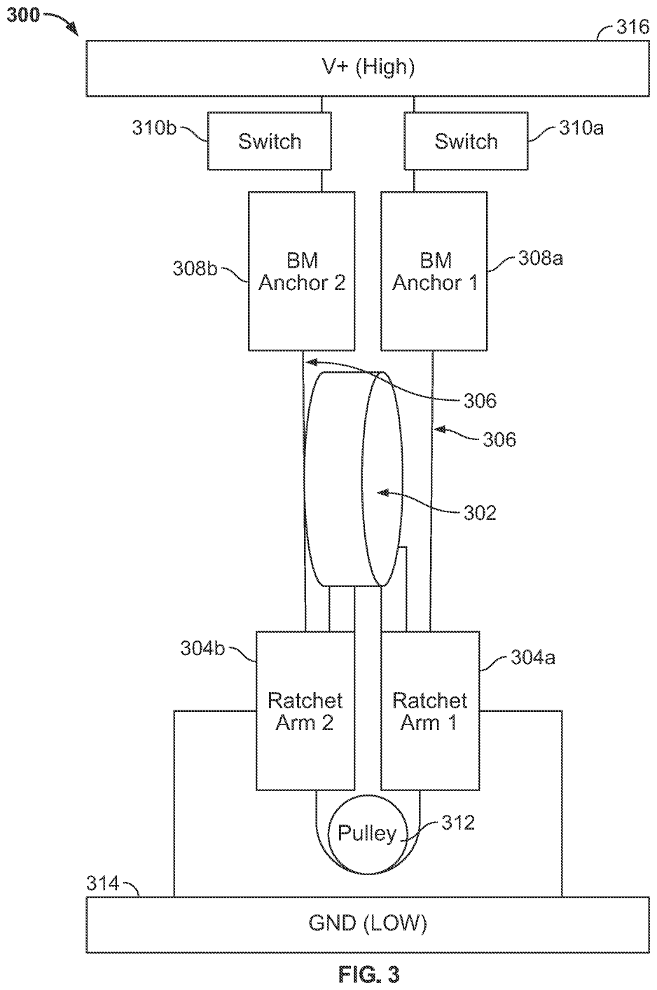

FIG. 3 is a block diagram of a drive mechanism 300 of a liquid drug delivery device in accordance with aspects described herein. In one example, the drive mechanism 300 is operable for use with the systems 100, 200 of FIGS. 1 and 2A-2D. For example, the drive mechanism 300 may be included in the pump mechanism 224 of the system 200 (e.g., as drive mechanism 250).

The drive mechanism 300 includes at least one ratchet wheel 302, a first ratchet arm 304a, a second ratchet arm 304b, at least one biasing member 306, a first biasing member anchor 308a, a second biasing member anchor 308b, a first switch 310a, a second switch 310b, and a pulley 312 (which may or may not rotate about its axis). In one example, the at least one ratchet wheel 302 includes a single ratchet wheel having a single set of ratchet teeth. In other examples, the at least one ratchet wheel 302 includes two ratchet wheels which may be stacked next to each other to provide two sets of adjacent ratchet teeth. The first and second ratchet arms 304a, 304b are configured to engage with the ratchet teeth of the at least one ratchet wheel 302 to rotate the at least one ratchet wheel 302.

In examples where the at least one ratchet wheel 302 includes a single ratchet wheel, the ratchet arms 304a, 304b may have different arm lengths or may be configured with a linear offset. For example, the arm lengths of, or the linear offset between, the ratchet arms 304a, 304b may differ by half of the stroke length it takes to complete one full tooth ratchet. Alternatively, when the at least one ratchet wheel 302 includes two stacked ratchet wheels, the ratchet arms 304a, 304b may have arm lengths that are the same. The rotation of the stacked ratchet wheels may be offset by half of a tooth length. For example, the offset of the second ratchet wheel may correspond to 360 degrees divided by twice the number of ratchet teeth in the first wheel.

The first and second ratchet arms 304a, 304b are coupled to the biasing member 306. In one example, the biasing member 306 corresponds to at least one SMA wire. The switches 310a, 310b may be operated to control the ratchet arms 304a, 304b via the biasing member 306. For example, the ratchet arms 304a, 304b may each be coupled to the biasing member 306 and a ground bus (or rail) 314. Likewise, the biasing member 306 may be coupled to a positive voltage bus 316. When the first switch 310a is turned on (e.g., closed), the biasing member 306 is pulled in a manner that causes the first ratchet arm 304a to contact the ratchet teeth of the at least one ratchet wheel 302. In some embodiments, when the first switch 310a is turned on, the biasing member 306 may contract in a manner that causes the first ratchet arm 304a to contact the ratchet teeth of the at least one ratchet wheel 302. The movement of the biasing member 306 around the pulley 312 causes the second ratchet arm 304b to pull away from the at least one ratchet wheel 302. In some examples, the second switch 310b is turned off (e.g., opened) while the first switch 310a is turned on. Similarly, when the second switch 310b is turned on (e.g., closed), the biasing member 306 is pulled (or contracts) in a manner that causes the second ratchet arm 304b to contact the ratchet teeth of the at least one ratchet wheel 302. The movement of the biasing member 306 around the pulley 312 causes the first ratchet arm 304a to pull away from the at least one ratchet wheel 302. In some examples, the first switch 310a is turned off (e.g., opened) while the second switch 310b is turned on. In one example, the anchors 308a, 308b provide a connection from the voltage bus 316 (or a PCB/power circuit coupled to the voltage bus 316) to the biasing member 306. The anchors 308a, 308b provide rigid points for the biasing member 306 to contract towards, pulling the ratchet arms 304a, 304b towards the ratchet wheel 302. It should be appreciated that the pulley 312 may be any mechanism that generates opposing motion on the ratchet arms 304a, 304b.

The drive mechanism 300 may be operated to convert the linear motion of the biasing member 306 into the rotational motion of the ratchet wheel(s) 302. For example, the biasing member 306 may be controlled (e.g., via the switches 310a, 310b) to alternate between causing the first ratchet arm 304a to contact the ratchet teeth of the at least one ratchet wheel 302 and causing the second ratchet arm 304b to contact the ratchet teeth of the at least one ratchet wheel 302. Alternating the drive mechanism 300 between the different states results in an incremental rotation of the ratchet wheel(s). As such, by coupling the ratchet wheel(s) 302 to a pumping mechanism (e.g., pump mechanism 224), the incremental rotational motion can be used to actuate the pump and deliver a liquid drug to a patient. For example, a pump mechanism coupling may be operable to connect the ratchet wheel(s) 302 to a drive element (e.g., the drive element 252 of FIG. 2C). The drive element may include a lead screw (or tube nut) coupled to a plunger (e.g., the plunger 202 of FIG. 2C). The drive mechanism 300 is operable to rotate the lead screw via the drive element and move the plunger to expel a liquid drug from a reservoir, pump chamber, or the like (e.g., the reservoir 225 of FIG. 2C).

In one example, the single or stacked ratchet wheel arrangement of the drive mechanism 300 reduces the overall size of the drive mechanism (or the drug delivery device). In some examples, the single or stacked ratchet wheel arrangement allows the drive mechanism 300 to be orientated with different options (e.g., vertical, horizontal, or angled). In addition, the biasing member 306 may operate with reduced travel when providing the ratcheting motion of the ratchet arms 304a, 304b.

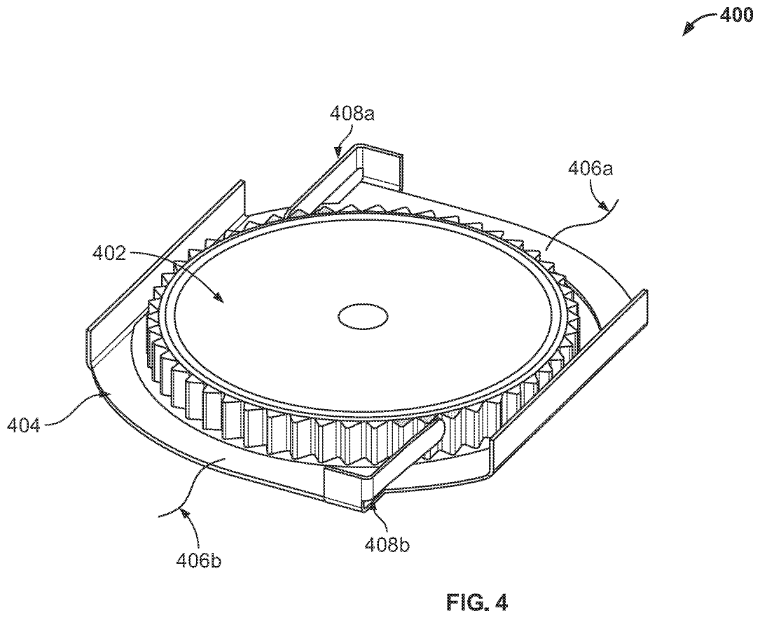

FIG. 4 illustrates a ratchet mechanism 400 in accordance with aspects described herein. The ratchet mechanism 400 includes a ratchet wheel 402, a ratchet arm 404, and a biasing member 406. In one example, the ratchet mechanism 400 is configured to be included in the drive mechanism 250 of FIG. 2C, 2D or the drive mechanism 300 of FIG. 3. For example, the ratchet wheel 402 may correspond to the ratchet wheel 302, the ratchet arm 404 may correspond to the ratchet arms 304a, 304b, and the biasing member 406 may correspond to the biasing member 306.

In one example, the ratchet wheel 402 includes a plurality of ratchet teeth. The ratchet arm 404 includes a first prong 408a and a second prong 408b. The ratchet arm 404 may be constructed from plastic materials (e.g., molded plastic), metal materials (e.g., sheet metal), or any other suitable materials. The ratchet arm 404 may be linearly constrained (i.e. may be constrained to only linear movement, in particular only linear movement along only one axis) via a ratchet arm constraining structure (not shown) while the ratchet wheel 402 is free to rotate around its fixed center axis.

The first prong 408a is operable interact with a ratchet tooth of the plurality of ratchet teeth to rotate the ratchet wheel 402 in a circular direction (e.g., clockwise). The second prong 408b is operable to interact with another ratchet tooth of the plurality of ratchet teeth to further rotate the ratchet wheel 402 in the circular direction. In one example, the biasing member 406 is coupled to the ratchet arm 404. The biasing member 406 is operable to move the first prong 408a of the ratchet arm in a first substantially linear direction (e.g., left, up, etc.) and to subsequently move the second prong 408b in a second substantially linear direction (e.g., right, down, etc.). In some examples, the second substantially linear direction is opposite the first substantially linear direction, enabling the rotation of the ratchet wheel in the circular direction.

In one example, the biasing member 406 includes separate biasing members 406a, 406b. For example, the biasing member 406 may include two different SMA wires, where a first SMA wire (e.g., biasing member 406a) is operable to move the ratchet arm 404 in the first linear direction and a second SMA wire (e.g., biasing member 406b) is operable to move the ratchet arm 404 in the second linear direction. Alternatively, the biasing member 406 may include two different types of biasing members. For example, the biasing member 406a may include an SMA wire operable to move the ratchet arm 404 in the first linear direction and the biasing member 406b may include a spring operable to move the ratchet arm 404 in the second linear direction.

The ratchet mechanism 400 may be configured such that number of ratchets (i.e. the number of interactions of the first prong 408a and second prong 408b) per revolution of the ratchet wheel 402 is double the number of teeth in the ratchet wheel 402. For example, the length of the first prong 408a may be half of a stroke longer than the length of the second prong 408b, allowing the prongs 408a, 408b to ratchet out of sequence. Ratcheting out of sequence creates an out-of-phase ratcheting effect that allows one prong (e.g., the first prong 408a) to rotate the ratchet wheel 402 just over half of a ratchet tooth while the other prong (e.g., the second prong 408b) retracts. This rotation amount is enough to engage the opposing ratchet prong to fall off the ratchet tooth that it is resting on, allowing the ratchet prong to push back towards the first until it has fallen off the next ratchet tooth. In other words, the rotation amount is enough to move the ratchet tooth the opposing ratchet prong was previously engaging far enough, that upon the opposing ratchet prong returning to its previous position (i.e. back in contact with the ratchet wheel 402) it will engage a the next rachet tooth. This sequence may repeat to provide a continuous incremental rotation of the ratchet wheel 402.

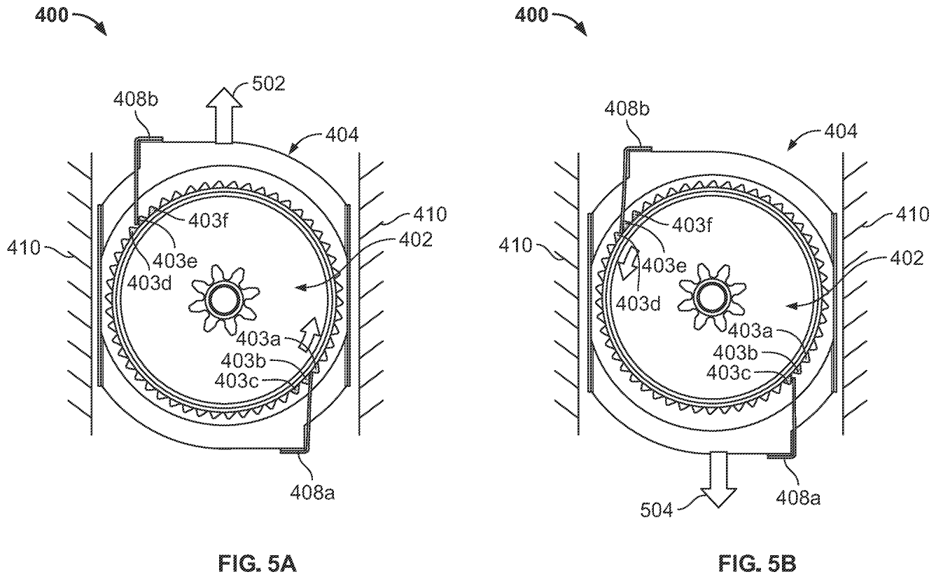

FIGS. 5A and 5B illustrate an example operation of the ratchet mechanism 400. In one example, FIG. 5A represents a first operational state of the drive mechanism 400 and FIG. 5B represents a second operational state of the drive mechanism 400.

As shown in FIG. 5A, in the first operational state, the ratchet arm 404 is pulled (e.g., via biasing member 406) in the first linear direction 502. The movement of the ratchet arm 404 in the first linear direction 502 causes the first prong 408a to contact a first tooth 403a of the plurality of ratchet teeth 403 of the ratchet wheel 402, while the second prong 408b disengages from the ratchet wheel 402. The force applied by the first prong 408a causes the ratchet wheel 402 to rotate about its fixed center axis in the circular direction. In some examples, the ratchet arm constraining structure 410 is operable to maintain movement of the ratchet arm 404 (or the prongs 408a, 408b) in a back and forth linear direction with respect to the ratchet wheel 402. The ratchet arm constraining structure 410 may include linear alignment features (or guides) that are positioned on opposite sides of the ratchet arm 404.

As shown in FIG. 5B, in the second operational state, the ratchet arm 404 is pulled (e.g., via biasing member 406) in the second linear direction 504. The movement of the ratchet arm 404 in the second linear direction 504 causes the second prong 408b to contact a second tooth 403d of the plurality of ratchet teeth of the ratchet wheel 402, while the first prong 408a disengages from the ratchet wheel 402. The force applied by the second prong 408b causes the ratchet wheel 402 to further rotate about its fixed center axis in the circular direction. The ratchet arm constraining structure 410 is operable to guide the ratchet arm 404 when transitioning from travel in the first linear direction 502 to travel in the second linear direction 504.

The ratchet mechanism 400 is operated to convert the linear motion of the biasing member(s) 406 into the rotational motion of the ratchet wheel 402. Alternating the ratchet mechanism 400 between the first and second operational states results in an incremental rotation of the ratchet wheel 402. As such, by coupling the ratchet wheel 402 to a pumping mechanism (e.g., pump mechanism 224), the incremental rotational motion can be used to actuate the pump and deliver a liquid drug to a patient. For example, a pump mechanism coupling may be operable to connect the ratchet wheel 402 to a drive element (e.g., drive element 252 of FIG. 2C). The drive element may include a lead screw (or tube nut) coupled to a plunger (e.g., the plunger 202 of FIG. 2C). The ratchet mechanism 400 is operable to rotate the lead screw via the drive element and move the plunger to expel a liquid drug from a reservoir, pump chamber, or the like (e.g., the reservoir 225 of FIG. 2C).

As shown in FIGS. 4-5B, the ratchet arm 404 may surround the circumference of the ratchet wheel 402. The circular opening of the ratchet wheel 404 can be configured to accommodate any desired size or thickness of the ratchet wheel 402. In other examples, rather than extending around the entire circumference of the ratchet wheel 402, the ratchet arm 404 may be configured differently.

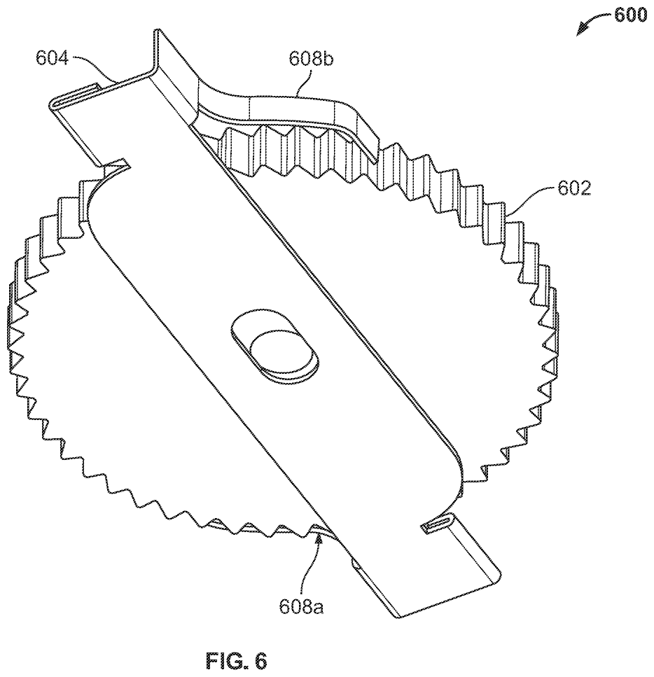

FIG. 6 illustrates a ratchet mechanism 600 in accordance with aspects described herein. The ratchet mechanism 600 includes a ratchet wheel 602 and a ratchet arm 604. In one example, the ratchet mechanism 600 is configured to be included in the drive mechanism 250 of FIG. 2C, 2D or the drive mechanism 300 of FIG. 3. For example, the ratchet wheel 602 may correspond to the ratchet wheel 302 and the ratchet arm 604 may correspond to the ratchet arms 304a, 304b. While not shown, the ratchet mechanism 600 may include one or more biasing members similar to the biasing member 306 of FIG. 3 and the biasing member(s) 406 of FIG. 4.

In one example, the ratchet wheel 602 includes a plurality of ratchet teeth. The ratchet arm 604 includes a first prong 608a and a second prong 608b. The ratchet mechanism 600 is substantially similar to the ratchet mechanism 400 of FIG. 4, except the ratchet arm 604 is nested under or over the ratchet wheel 602. In one example, the ratchet arm 604 has a length that extends along a diameter of the ratchet wheel 602 and protrudes from opposite sides of the ratchet wheel 602 beyond the plurality of ratchet teeth. In some examples, each respective end of the ratchet arm 604 has a biasing mechanism engagement point and a respective initial point for one of either the first prong 608a or the second prong 608b. Further, as shown in FIG. 6, the ratchet arm 604 may comprise an oblong hole. The ratchet arm 604 may be connected to the ratchet wheel 602 by a shaft of the ratchet wheel 602 being disposed within the oblong hole.

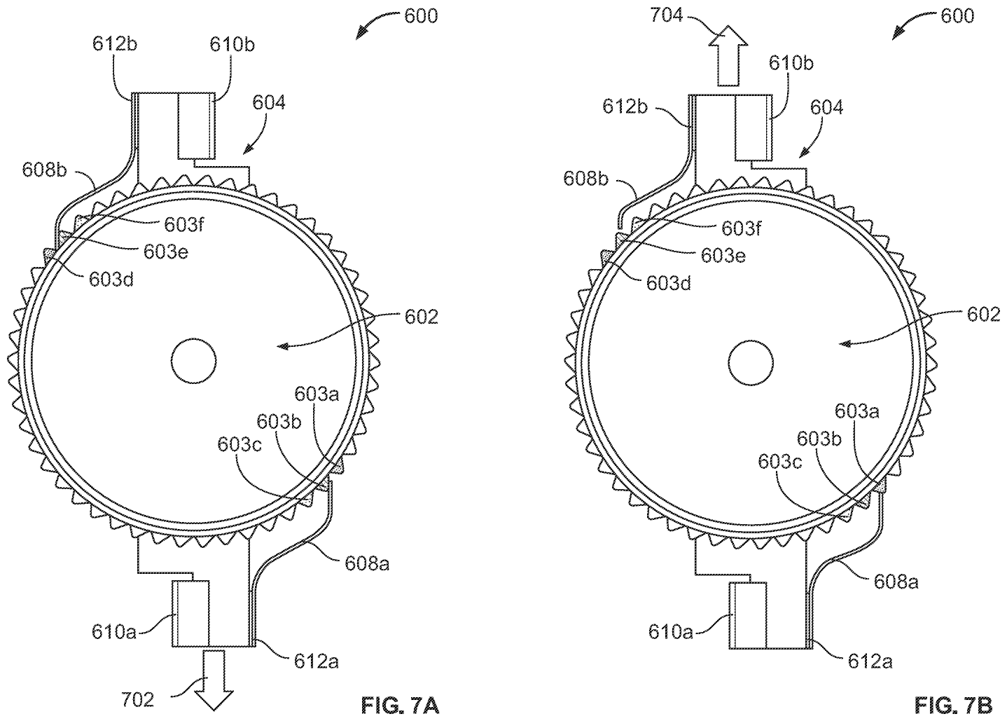

FIGS. 7A and 7B illustrate an example operation of the ratchet mechanism 600. In one example, FIG. 7A represents a first operational state of the drive mechanism 600 and FIG. 7B represents a second operational state of the drive mechanism 600.

As shown in FIG. 7A, in the first operational state, the ratchet arm 604 is pulled (e.g., via a biasing member) in a first linear direction 702. In one example, each respective end of the ratchet arm 604 includes a biasing mechanism engagement point and an initial prong point. For example, the ratchet arm 604 includes a first end having a first biasing mechanism engagement point 610a and an initial prong point 612a for the first prong 608a. Likewise, the ratchet arm 604 includes a second end having a second biasing mechanism engagement point 610b and an initial prong point 612b for the second prong 608b.

The movement of the ratchet arm 604 in the first linear direction 702 causes the second prong 608b to contact a first tooth 603d of the plurality of ratchet teeth 603 of the ratchet wheel 602, while the first prong 608a disengages from the ratchet wheel 602. The force applied by the second prong 608b causes the ratchet wheel 602 to rotate about its fixed center axis in the circular direction.

As shown in FIG. 7B, in the second operational state, the ratchet arm 604 is pulled (e.g., via a biasing member) in a second linear direction 704. The movement of the ratchet arm 604 in the second linear direction 704 causes the first prong 608a to contact a second tooth 603a of the plurality of ratchet teeth 603 of the ratchet wheel 602, while the second prong 608b disengages from the ratchet wheel 602. The force applied by the first prong 608a causes the ratchet wheel 602 to further rotate about its fixed center axis in the circular direction.

While not shown, a ratchet arm constraining structure may be operable to maintain movement of the ratchet arm 604 (or the prongs 608a, 608b) in a back and forth linear direction with respect to the ratchet wheel 602. The ratchet arm constraining structure may include linear alignment features (or guides) that are positioned at the top and bottom tabs of the ratchet arm 604 that extend beyond the diameter of the ratchet wheel 602.

The ratchet mechanism 600 is operated to convert the linear motion of the biasing member(s) into the rotational motion of the ratchet wheel 602. Alternating the ratchet mechanism 600 between the first and second operational states results in an incremental rotation of the ratchet wheel 602. As such, by coupling the ratchet wheel 602 to a pumping mechanism (e.g., pump mechanism 224), the incremental rotational motion can be used to actuate the pump and deliver a liquid drug to a patient. For example, a pump mechanism coupling may be operable to connect the ratchet wheel 602 to a drive element (e.g., drive element 252 of FIG. 2C). The drive element may include a lead screw (and optionally a tube nut over the lead screw) coupled to a plunger (e.g., the plunger 202 of FIG. 2C). The ratchet mechanism 600 is operable to rotate the lead screw via the drive element and move the plunger to expel a liquid drug from a reservoir, pump chamber, or the like (e.g., the reservoir 225 of FIG. 2C).

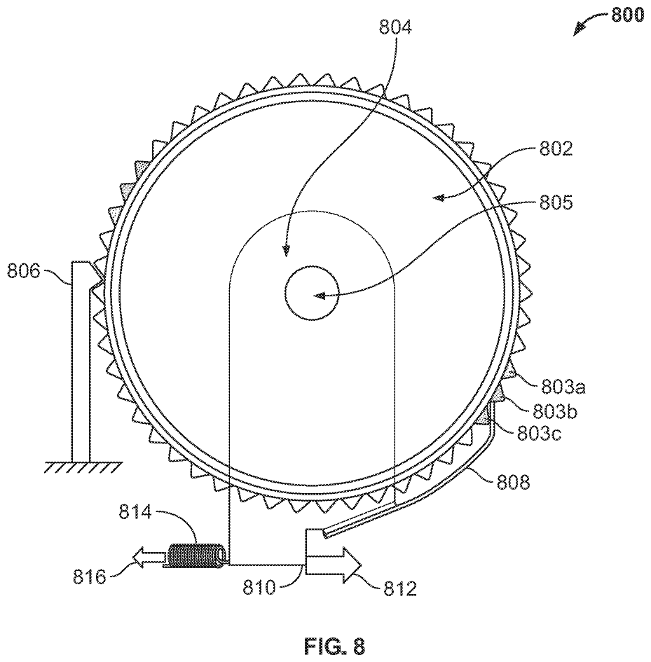

FIG. 8 illustrates a ratchet mechanism 800 in accordance with aspects described herein. The ratchet mechanism 800 includes a ratchet wheel 802, a ratchet arm 804, and a stop pawl 806. In one example, the ratchet mechanism 800 is configured to be included in the drive mechanism 250 of FIGS. 2C, 2D.

In one example, the ratchet wheel 802 includes a plurality of ratchet teeth 803. The ratchet arm 804 extends from a center point 805 of the ratchet wheel 802 and beyond a perimeter of the ratchet wheel 802. The ratchet arm 804 may be constructed from plastic materials (e.g., molded plastic), metal materials (e.g., sheet metal), or any other suitable materials. The ratchet arm 804 includes a prong 808 operable to interact with the plurality of teeth 803 of the ratchet wheel 802 and rotate the ratchet wheel 802 in a circular direction. In some examples, the ratchet arm 804 includes a biasing member attachment point 810 that is farther from the center point 805 of the ratchet wheel 802 than the prong 808 and operable to attach a biasing member (not shown) to the ratchet arm 804. In some examples, the ratio of the two distances (i.e. the distance between the biasing member 810 and the center point 805 and the distance between the prong 808 and the center point 805) corresponds to a mechanical advantage that is added to the ratchet mechanism 800.

In one example, the ratchet mechanism 800 is configured to operate as a rotary traveling ratchet. The ratchet arm 804 is pulled (e.g., via a biasing member at the biasing member attachment point 810) in a first linear direction 812. The term “being pulled in a linear direction” as used herein, may refer to a force in a linear direction being exerted on a part. The movement of the ratchet arm 804 in the first linear direction 812 causes the prong 808 to contact a first tooth 803b of the plurality of ratchet teeth 803 of the ratchet wheel 802. It should be noted that movement of the ratchet arm 804 in the first linear direction 812, relates to the ratchet arm 804 moving around the center point 805, in particular wherein at the biasing member attachment point 810 the ratchet moves in the first linear direction 812. The stop pawl 806 is disengaged from the ratchet wheel 802 in response to the prong 808 interacting with the ratchet wheel 804. The force applied by the prong 808 causes the ratchet wheel 802 to rotate about the center point 805 (e.g., its fixed center axis) in the circular direction. A spring 814 is operable to reset the ratchet arm 804 (or prong 808) by pulling the ratchet arm 604 in a second linear direction 816. In some examples, the second linear direction 816 is opposite the first linear direction 812. The stop pawl 806 is operable to engage the ratchet wheel 802 to prevent a reverse rotation of the ratchet wheel 802 after interaction by the prong 808.

The ratchet arm 804 may rotate about the center point 805 as it engages/disengages from the ratchet wheel 802. Since the rotation axes for the ratchet wheel 802 and ratchet arm 804 are aligned, the tolerances of the ratchet mechanism 800 may be reduced. In some examples, location of the biasing member attachment point 810 is aligned directly under the axis of rotation (e.g., the center point 805). In some embodiments, the biasing member 810 may be a torsion spring.

The ratchet mechanism 800 is operated to convert the linear motion of the biasing member(s) (e.g., coupled to the biasing member attachment point 810) into the rotational motion of the ratchet wheel 802. Operating the ratchet mechanism 800 results in an incremental rotation of the ratchet wheel 802. As such, by coupling the ratchet wheel 802 to a pumping mechanism (e.g., pump mechanism 224), the incremental rotational motion can be used to actuate the pump and deliver a liquid drug to a patient. For example, a pump mechanism coupling may be operable to connect the ratchet wheel 802 to a drive element (e.g., drive element 252 of FIG. 2C). The drive element may include a lead screw (or tube nut) coupled to a plunger (e.g., the plunger 202 of FIG. 2C). The ratchet mechanism 800 is operable to rotate the lead screw via the drive element and move the plunger to expel a liquid drug from a reservoir, pump chamber, or the like (e.g., the reservoir 225 of FIG. 2C).

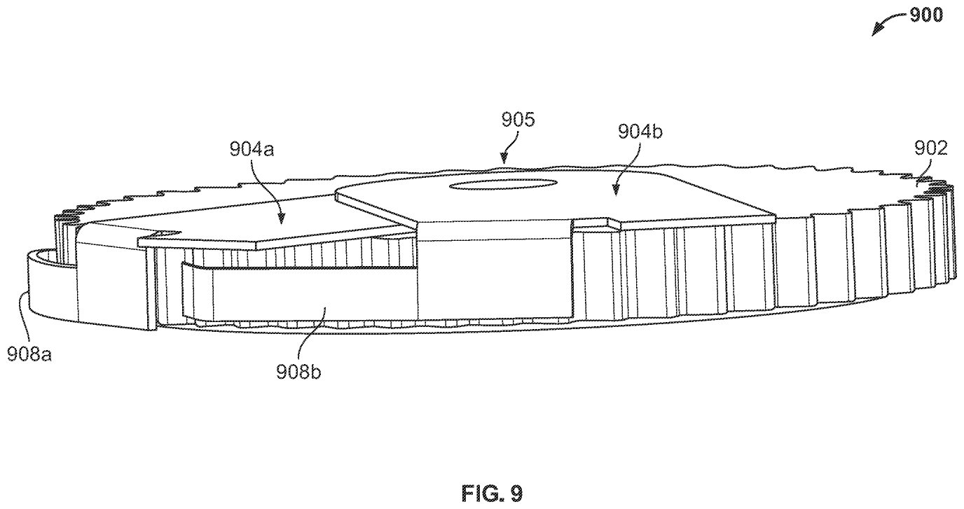

FIG. 9 illustrates a ratchet mechanism 900 in accordance with aspects described herein. The ratchet mechanism 900 includes a ratchet wheel 902, a first ratchet arm 904a, and a second ratchet arm 904b. In one example, the ratchet mechanism 900 is configured to be included in the drive mechanism 250 of FIGS. 2C, 2D.

In one example, the ratchet wheel 902 includes a plurality of ratchet teeth. The first and second ratchet arms 904a, 904b extend from a center point 905 of the ratchet wheel 902 and beyond a perimeter of the ratchet wheel 902. The first ratchet arm 904a includes a first prong 908a and the second ratchet arm 904b includes a second prong 908b. The ratchet arms 904a, 904b may be constructed from plastic materials (e.g., molded plastic), metal materials (e.g., sheet metal), or any other suitable materials. In some examples, the ratchet arms 904a, 904b are substantially identical. The ratchet wheels 904a, 904b may be attached to the front or back of the ratchet wheel 902 and operable to run (e.g., travel) next to each other.

The first prong 908a of the first ratchet arm 904a is operable interact with a ratchet tooth of the plurality of ratchet teeth to rotate the ratchet wheel 902 in a circular direction (e.g., clockwise). The second prong 908b is operable to interact with another ratchet tooth of the plurality of ratchet teeth to further rotate the ratchet wheel 902 in the circular direction. In one example, a biasing member is coupled to each ratchet arm 904. For example, each ratchet arm 904a, 904b may include a biasing member attachment point that is farther from the center point 905 of the ratchet wheel 902 than the prongs 908a, 908b. The biasing member attachment points are operable to attach a biasing member (not shown in this example) to each ratchet arm 904a, 904b.

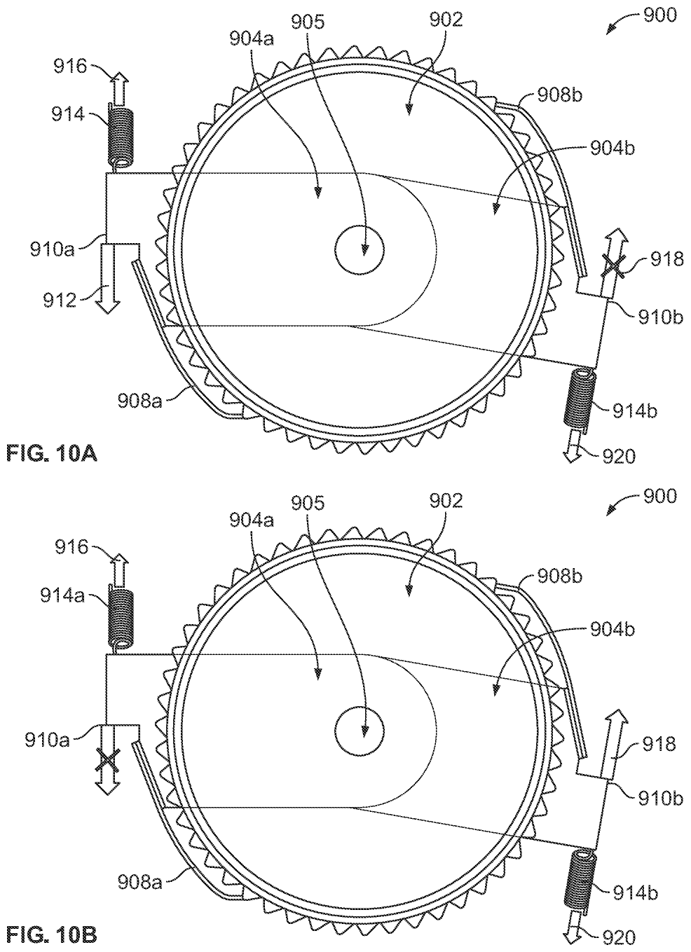

FIGS. 10A and 10B illustrate an example operation of the drive mechanism 900. In one example, FIG. 10A represents a first operational state of the drive mechanism 900 and FIG. 10B represents a second operational state of the drive mechanism 900. In one example, a first biasing member may be operable to pull the first ratchet arm 904a (and the first prong 908a) around the ratchet wheel 902 and a second biasing member may be operable to pull the second ratchet arm 904b (and the second prong 908b) around the ratchet wheel 902. The first and second biasing members are operated such that the first ratchet arm 904a and the second ratchet arm 904b engage with the ratchet wheel 902 in an alternating sequence. In some examples, the spacing between the first ratchet arm 904a and the second ratchet arm 904b corresponds to 360 degrees divided by twice the number of ratchet teeth in the ratchet wheel 902. The first biasing member and the second biasing member may be SMA wires, springs, elastic bands, or the like.

As shown in FIG. 10A, in the first operational state, the first ratchet arm 904a is pulled (e.g., via a first biasing member at the biasing member attachment point 910a) in a first linear direction 912. The movement of the ratchet arm 904a in the first linear direction 912 causes the first prong 908a to contact a first tooth of the plurality of ratchet teeth of the ratchet wheel 902. The force applied by the first prong 908a causes the ratchet wheel 902 to rotate about the center point 905 (e.g., its fixed center axis) in a circular direction (e.g., counter-clockwise). A spring 914a coupled to the ratchet arm 904a (e.g., opposite the biasing member attachment point 910a) is operable to reset the ratchet arm 904a (or prong 908a) by pulling the ratchet arm 904a in a second linear direction 916. In some examples, the second linear direction 916 is opposite the first linear direction 914.

During the first operational state, the second ratchet arm 904b remains stationary. In some examples, the second ratchet arm 904b provides functionality similar to a pawl mechanism (e.g., stop pawl 806). The second ratchet arm 904b is operable to engage the ratchet wheel 902 to prevent a reverse rotation of the ratchet wheel 902 after interaction by the first prong 908a. It should be appreciated that the second ratchet arm 904b is disengaged from the ratchet wheel 902 in response to the first prong 908a interacting with the ratchet wheel 902, allowing forward rotation of the ratchet wheel 902. In some embodiments, the ratchet arm 904b is disengaged from the rachet wheel 902 due to the first biasing member relaxing (i.e. exerting less or no force on the biasing member attachment point 910a), leading to the force exerted by the spring 914 resulting in the ratchet arm 904 being pulled in the second linear direction 916.

As shown in FIG. 10B, in the second operational state, the second ratchet arm 904b is pulled (e.g., via a second biasing member at the biasing member attachment point 910b) in a third linear direction 918. The movement of the ratchet arm 908b in the third linear direction 918 causes the second prong 908b to contact a second tooth of the plurality of ratchet teeth of the ratchet wheel 902. The force applied by the second prong 908b causes the ratchet wheel 902 to further rotate about the center point 905 in the circular direction. A spring 914b coupled to the ratchet arm 904b (e.g., opposite the biasing member attachment point 901b) is operable to reset the ratchet arm 904b (or prong 908b) by pulling the ratchet arm 904b in a fourth linear direction 920. In some examples, the fourth linear 920 direction is opposite the third linear direction 918. In some embodiments, the movement of the ratchet arm 904 in the first, second, third and fourth linear direction 912, 914, 918, 920 may relate to the ratchet arm 904 moving around the center point 905.

During the second operational state, the first ratchet arm 904a remains stationary. In some examples, the first ratchet arm 904a provides functionality similar to a pawl mechanism. The first ratchet arm 904a is operable to engage the ratchet wheel 902 to prevent a reverse rotation of the ratchet wheel 902 after interaction by the second prong 908b. It should be appreciated that the first ratchet arm 904a is disengaged from the ratchet wheel 902 in response to the second prong 908b interacting with the ratchet wheel 902, allowing forward rotation of the ratchet wheel 902.

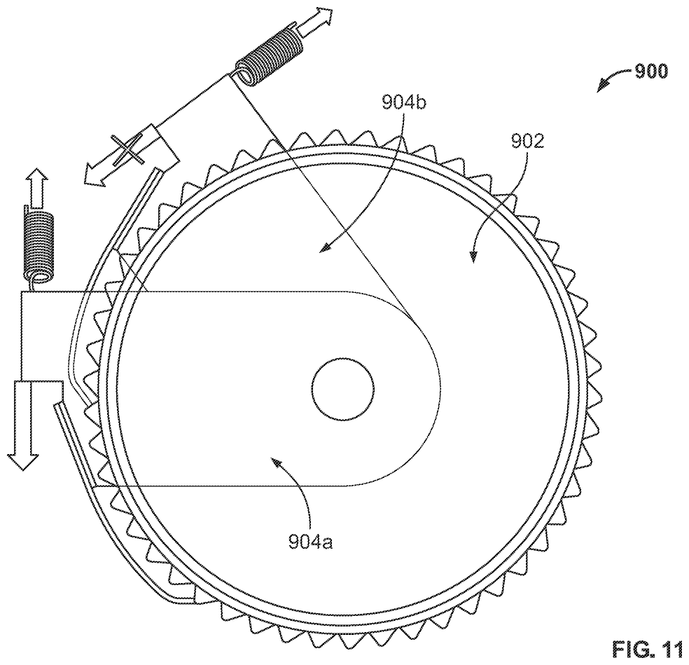

As shown in FIG. 11, the first and second ratchet arms 904a, 904b may be positioned with a nested (or partially overlapping) arrangement. In some examples, such an arrangement may further reduce the size of the ratchet mechanism 900 (or the drive mechanism). In some examples, the ratchet mechanism 900 can include more than two ratchet arms. For example, the ratchet mechanism 900 may include a third traveling ratchet arm. The third ratchet mechanism may increase the number of ratchets per revolution of the ratchet wheel 902 to triple the number of ratchet teeth (e.g., each ratchet arm may engage with each tooth of the ratchet wheel 902 to mimic the effect of tripling the number of ratchet teeth).

The ratchet mechanism 900 is operated to convert the linear motion of the biasing member(s) (e.g., coupled to the biasing member attachment points 910a, 910b) into the rotational motion of the ratchet wheel 902. Alternating the ratchet mechanism 900 between the first and second operational states results in an incremental rotation of the ratchet wheel 902. As such, by coupling the ratchet wheel 902 to a pumping mechanism (e.g., pump mechanism 224), the incremental rotational motion can be used to actuate the pump and deliver a liquid drug to a patient. For example, a pump mechanism coupling may be operable to connect the ratchet wheel 902 to a drive element (e.g., drive element 252 of FIG. 2C). The drive element may include a lead screw (or tube nut) coupled to a plunger (e.g., the plunger 202 of FIG. 2C). The ratchet mechanism 900 is operable to rotate the lead screw via the drive element and move the plunger to expel a liquid drug from a reservoir, pump chamber, or the like (e.g., the reservoir 225 of FIG. 2C).

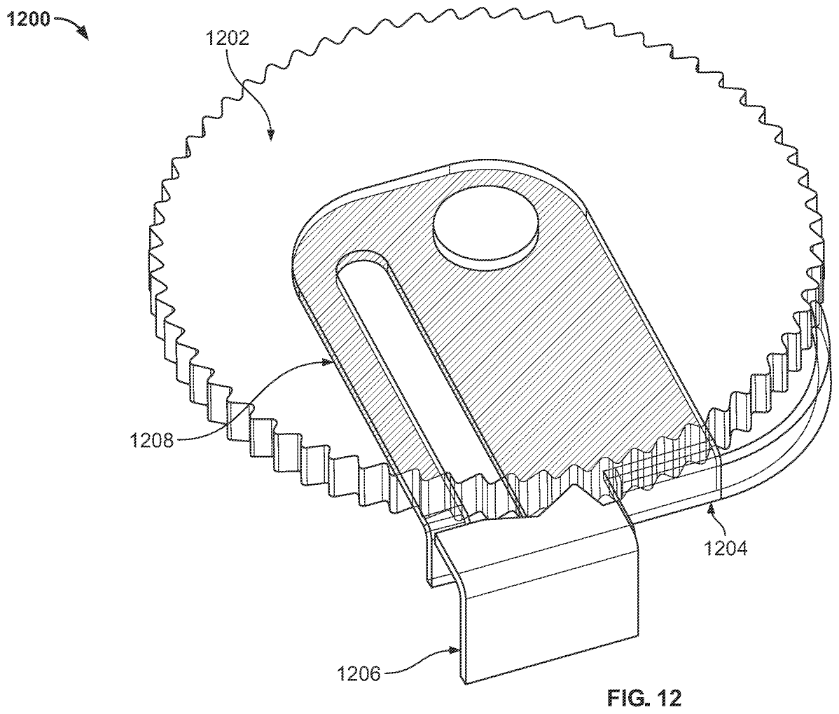

FIG. 12 illustrates a ratchet mechanism 1200 in accordance with aspects described herein. The ratchet mechanism 1200 is substantially similar to the ratchet mechanism 800 of FIG. 8, except the ratchet mechanism 1200 includes a beam spring 1208.

In one example, the stop pawl 1206 is combined with a contact surface for the built-in beam spring 1206. The beam spring 1206 runs along a side of the ratchet arm 1204 (e.g., the left side). As the ratchet arm 904 pushes the ratchet wheel 1202, the ratchet tooth that is engaged with the stop pawl 1206 pushes the pawl 1206 away from the ratchet wheel 1202 until it falls off the next (e.g., adjacent) tooth. The beam spring 1206 includes an intentional interference that maintains the ratchet arm 1204 with a slight back torque. In some examples, the ratchet arm 1204 includes a hard stop feature that interacts with the stop pawl 1206 to provide a repeatable starting position.

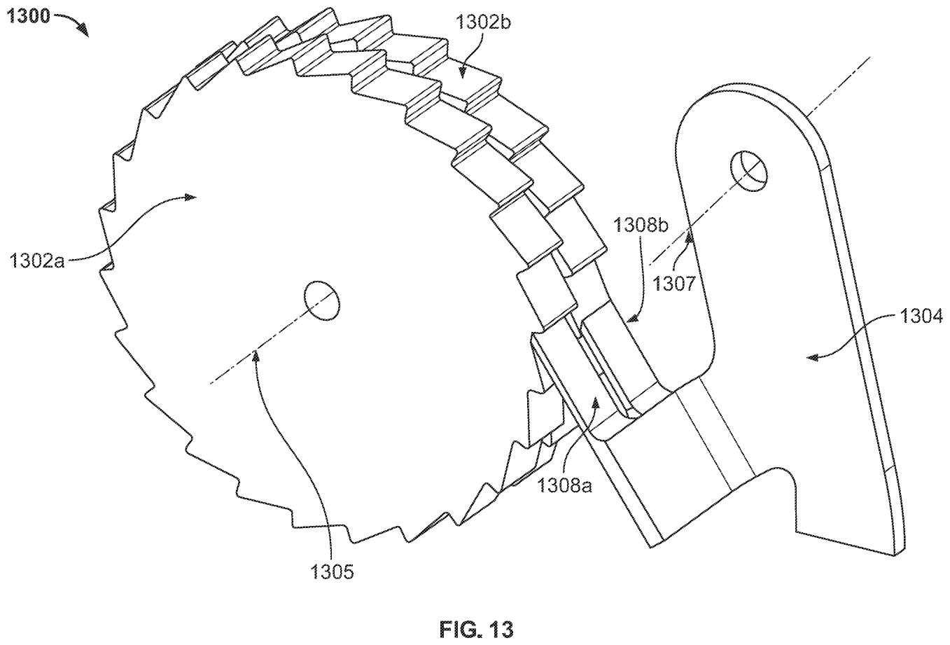

FIG. 13 illustrates a ratchet mechanism 1300 in accordance with aspects described herein. The ratchet mechanism 1300 includes a first ratchet wheel 1302a, a second ratchet wheel 1302b, and a ratchet arm 1304. In one example, the ratchet mechanism 800 is configured to be included in the drive mechanism 250 of FIGS. 2C, 2D.

The first ratchet wheel 1302a includes a first plurality of ratchet wheel teeth and is centered about a wheel axis 1305. The second ratchet wheel 1302b includes a second plurality of ratchet wheel teeth centered about the wheel axis 1305. In some examples, the first and second ratchet wheels 1302a, 1302b may correspond to a single ratchet wheel having two sets of ratchet teeth. In some embodiments, the first and second ratchet wheels 1302a, 1302b may correspond to two individual rachet wheels, each having a set of ratchet teeth, wherein the two ratchet wheels are mechanically coupled, e.g. by pinning. The ratchet arm 1304 includes a first prong 1308a operable to interact with the first plurality of ratchet wheel teeth of the first ratchet wheel 1304a and rotate the first ratchet wheel 1302a in a circular direction (e.g., clockwise). Likewise, the ratchet arm 1304 includes a second prong 1308b operable to interact with the second plurality of ratchet wheel teeth of the second ratchet wheel 1304b and rotate the second ratchet wheel 1302b in the circular direction. In one example, the ratchet arm 1304 is operable to rotate about an arm axis 1307 positioned outside the radius of the first and second ratchet wheels 1302a, 1302b. Wheel axis 1305 and arm axis 1307 may be offset in parallel to each other. The ratchet arm 1304 may be constructed from plastic materials (e.g., molded plastic), metal materials (e.g., sheet metal), or any other suitable materials. In other examples, the ratchet arm 1304 may correspond to two separate ratchet arms (e.g., each having one prong).

The ratchet arm 1304 may include a biasing member attachment point that is operable to attach one or more biasing members (not shown) to the ratchet arm 1304. In one example, the biasing member attachment point is positioned at the bottom portion of the ratchet arm 1304. In some examples, a biasing member (e.g., one or more SMA wires and/or a spring) is operable to move the ratchet arm 1304 in a first linear direction and/or a second linear direction via the biasing member attachment point. The movement of the ratchet arm 1304 in the first and second linear directions causes the ratchet arm 1307 to rotate (or swing) about the arm axis 1307. In other examples, multiple biasing members are operable to move the ratchet arm 1304 via the biasing member attachment point. For example, a first SMA wire may be operable to move the ratchet arm 1304 in the first linear direction and a second SMA wire may be operable to move the ratchet arm 1304 in the second linear direction. Alternatively, two different types of biasing members may be used. For example, an SMA wire may be operable to move the ratchet arm 1304 in the first linear direction and a spring may be operable to move the ratchet arm 1304 in the second linear direction.

The ratchet arm 1304 is pulled (e.g., via a biasing member at the biasing member attachment point) and the movement of the ratchet arm 1304 causes the first prong 1308a to contact a tooth of the first plurality of ratchet teeth of the first ratchet wheel 1302a and the second prong 1308b to contact a tooth of the second plurality of ratchet teeth of the second ratchet wheel 1302b. The forces applied by the prongs 1308a, 1308b causes the ratchet wheels 1302a, 1302b to rotate about the wheel axis 1305 in the circular direction. In some examples, the first plurality of ratchet teeth of the first ratchet wheel 1302a are offset (e.g., by half of a ratchet tooth length) from the second plurality of ratchet teeth of the second ratchet wheel 1302b. In some examples, elongating the lengths of the ratchet wheel teeth may allow the overall wheel size (or diameter) to be reduced while still achieving a given number of ratchets per revolution. In an alternative example, different length ratchet arms may be used with a single ratchet tooth set to achieve a similar effect.

The ratchet mechanism 1300 is operated to convert the linear motion of the biasing member(s) (e.g., coupled to ratchet arm 1304) into the rotational motion of the ratchet wheels 1302a, 1302b. Alternating the ratchet mechanism 1300 between the first and second operational states results in an incremental rotation of the ratchet wheels 1302a, 1302b. As such, by coupling the ratchet wheels 1302a, 1302b to a pumping mechanism (e.g., pump mechanism 224), the incremental rotational motion can be used to actuate the pump and deliver a liquid drug to a patient. For example, a pump mechanism coupling may be operable to connect the ratchet wheels 1302a, 1302b to a drive element (e.g., drive element 252 of FIG. 2C). The drive element may include a lead screw (or tube nut) coupled to a plunger (e.g., the plunger 202 of FIG. 2C). The ratchet mechanism 1300 is operable to rotate the lead screw via the drive element and move the plunger to expel a liquid drug from a reservoir, pump chamber, or the like (e.g., the reservoir 225 of FIG. 2C).

As described above, improved drive mechanisms for liquid drug delivery devices are provided herein. In at least one embodiment, the drive mechanism includes a ratchet wheel including a plurality of teeth and a ratchet arm including a first prong and a second prong. In some examples, the first prong is operable to interact with a tooth of the plurality of teeth to rotate the ratchet wheel in a circular direction and the second prong is operable to interact with another tooth of the plurality of teeth to further rotate the ratchet wheel in the first circular direction. Such an arrangement enables the drive mechanism to have a compact form factor.

The techniques described herein for a drug delivery system (e.g., the system 100, the system 200, or any components thereof) may be implemented in hardware, software, or any combination thereof. Any component as described herein may be implemented in hardware, software, or any combination thereof. For example, the systems 100, 200 or any components thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Certain examples of the present disclosed subject matter were described above. It is, however, expressly noted that the present disclosed subject matter is not limited to those examples, but rather the intention is that additions and modifications to what was expressly described herein are also included within the scope of the disclosed subject matter. Moreover, it is to be understood that the features of the various examples described herein were not mutually exclusive and may exist in various combinations and permutations, even if such combinations or permutations were not made express herein, without departing from the spirit and scope of the disclosed subject matter. In fact, variations, modifications, and other implementations of what was described herein will occur to those of ordinary skill in the art without departing from the spirit and the scope of the disclosed subject matter. As such, the disclosed subject matter is not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of example examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

1. A drug delivery drive mechanism, comprising:
a ratchet wheel including a plurality of teeth;
a ratchet arm including a first prong and a second prong, wherein the first prong is operable interact with a tooth of the plurality of teeth to rotate the ratchet wheel in a circular direction, and the second prong is operable to interact with another tooth of the plurality of teeth to further rotate the ratchet wheel in the circular direction.

2. The drug delivery drive mechanism of claim 1, further comprising:
a biasing member coupled to the ratchet arm, wherein:
the biasing member is operable to move the ratchet arm and the first prong in a first substantially linear direction, and
to subsequently move the ratchet arm and the second prong in a second substantially linear direction, opposite the first substantially linear direction, enabling the rotation of the ratchet wheel in the circular direction.

3. The drug delivery device mechanism of claim 2, wherein the biasing member comprises:
a pulley mechanism; and
a smart memory alloy wire operable to move the ratchet arm in the first linear direction.

4. The drug delivery drive mechanism of claim 2, wherein the biasing member comprises:
a first smart memory alloy wire operable to move the ratchet arm in the first linear direction, and
a second smart memory alloy wire operable to move the ratchet arm in the second linear direction.

5. The drug delivery drive mechanism of claim 2, wherein the biasing member comprises:
a first smart memory alloy wire operable to move the ratchet arm in the first linear direction, and
a spring operable to the ratchet arm in the second linear direction.

6. The drug delivery drive mechanism of claim 1, further comprising:
a ratchet arm constraining structure that is operable to maintain movement of the ratchet arm in a back and forth linear direction with respect to the ratchet wheel.

7. The drug delivery drive mechanism of claim 1, wherein the first prong is longer than the second prong.

8. The drug delivery drive mechanism of claim 7, wherein the first prong and the second prong provide an out-of-phase ratcheting effect.

9. The drug delivery drive mechanism of claim 1, wherein the ratchet arm comprises:
a ratchet arm length that extends along a diameter of the ratchet wheel and protrudes from opposite sides of the ratchet wheel beyond the number of teeth, with each respective end of the ratchet arm having a biasing mechanism engagement point and a respective initial point for one of either the first prong or the second prong.

10. The drug delivery drive mechanism of claim 1, further comprising:
a pump mechanism coupling operable to connect the ratchet wheel to a drive element.

11. The drug delivery drive mechanism of claim 10, wherein the drive element includes a lead screw coupled to a plunger, and the pump mechanism is operable to rotate the lead screw and move the plunger to expel a liquid drug from a reservoir.

12. A drug delivery drive mechanism, comprising:
a ratchet wheel including a plurality of teeth and having a center point;
a ratchet arm extending from the center point of the ratchet wheel and beyond a perimeter of the ratchet wheel; and
a biasing member operable to move the ratchet arm,
wherein the ratchet arm includes:
a prong operable to interact with a tooth of the plurality of teeth of the ratchet wheel and rotate the ratchet wheel in a circular direction, and
a biasing member attachment point that is farther from the center point of the ratchet wheel than the prong, and operable to attach the biasing member to the ratchet arm.

13. The drug delivery drive mechanism of claim 12, wherein the biasing member attachment point is aligned with the center point of the ratchet wheel.

14. The drug delivery drive mechanism of claim 12, further comprising:
a spring coupled to the ratchet arm, wherein the biasing member is operable to pull the ratchet arm in a first linear direction and the spring is operable to pull the ratchet arm in a different, second linear direction.

15. The drug delivery drive mechanism of claim 12, comprising:
a secondary ratchet arm extending from the center point of the ratchet wheel and beyond the perimeter of the ratchet wheel; and
a secondary biasing member operable to move the secondary ratchet arm,
wherein the secondary ratchet arm includes:
a secondary prong operable to interact with a different tooth of the plurality of teeth of the ratchet wheel and rotate the ratchet wheel in a circular direction, and
a secondary biasing member attachment point that is farther from the center point of the ratchet wheel than the secondary prong, and operable to attach the secondary biasing member to the ratchet arm.

16. The drug delivery drive mechanism of claim 15, further comprising:
a secondary spring coupled to the secondary ratchet arm, wherein the secondary biasing member is operable to pull the secondary ratchet arm in a first linear direction and the secondary spring is operable to pull the secondary ratchet arm in a different, second linear direction.

17. The drug delivery drive mechanism of claim 12, further comprising:
a stop pawl operable to engage the ratchet wheel to prevent a reverse rotation of the ratchet wheel after interaction by the prong.

18. The drug delivery drive mechanism of claim 17, wherein the ratchet arm further comprises:
a beam spring running along a side of the ratchet arm, wherein the stop pawl is disengaged from the ratchet wheel in response to the prong interacting with the ratchet wheel.

19. The drug delivery drive mechanism of claim 12, further comprising:
a pump mechanism coupling operable to connect the ratchet wheel to a drive element.

20. The drug delivery drive mechanism of claim 19, wherein the drive element includes a lead screw coupled to a plunger, and the pump mechanism is operable to rotate the lead screw and move the plunger to expel a liquid drug from a reservoir.
